(19) United States
(12) Patent Application Publication
(10) Pub. No.: US 2019/0117519 A1
(43) Pub. Date: Apr. 25, 2019

(54) ENTERAL DRUG DELIVERY SYSTEM

(71) Applicants: The Regents of the University of California, Oakland, CA (US); Politecnico Di Milano, Milano (IT)

(72) Inventors: Geert W. Schmid-Schonbein, La Jolla, CA (US); Frank A. Delano, La Jolla, CA (US); Federico Aletti, Milan (IT)

(21) Appl. No.: 16/094,715

(22) PCT Filed: Apr. 20, 2017
(86) PCT No.: PCT/US2017/028583

§ 371 Date: Oct. 18, 2018

Related U.S. Application Data
(60) Provisional application No. 62/326,443, filed on Apr. 22, 2016.

(51) Int. Cl. A61J 15/00; A61M 1/00

(52) U.S. Cl. CPC A61J 15/0073; A61M 2210/1057; A61J 15/0049; A61J 15/0076; A61J 15/0084; A61M 1/0025; A61M 2205/3324; A61M 2205/3344; A61M 2205/3334; A61M 2205/3368; A61M 2205/3327; A61M 2205/3303; A61M 2205/3306; A61M 2205/50; A61M 2205/18; A61M 2210/1053; A61J 15/0003

(57) ABSTRACT

Provided herein is an enteral safe feeding catheter that allows for a higher rate of delivering liquid food and/or drugs to the stomach and/or duodenum of a mammal while simultaneously allowing for aspiration of any regurgitated reflux escaping the stomach into the esophagus. Systems incorporating the catheter and methods of use thereof are also provided herein.

### CROSS REFERENCE TO RELATED APPLICATION(S)

This application claims the benefit of priority under 35 U.S.C. § 119(e) of U.S. Ser. No. 62/326,443, filed Apr. 22, 2016, the entire content of which is incorporated herein by reference.

### BACKGROUND OF THE INVENTION

### Field of the Invention

The present invention relates to improved enteral feeding catheters incorporating one or more environmental sensors for automated delivery of liquid food or drugs and safety of patients.

### Background Information

Numerous situations exist in which a body cavity needs to be catheterized to achieve a desired medical goal. One relatively common situation is to provide nutritional solutions or medicines directly into the stomach or intestines. Certain catheters are inserted into through the patient's nose or mouth for treating the gastrointestinal tract. These catheters, sometimes referred to as enteral catheters, typically include feeding tubes. The feeding tube lies in the stomach or intestines, and a feeding bag delivers liquid nutrient, liquid medicine or a combination of the two to the patient. When a feeding tube is inserted through a patient's nare, and it is determined that a patient cannot be fed into the stomach, a second tube is typically inserted through the patient's other nare.

However, enteral feeding and/or rapid infusion of liquid medicines is at risk to generate aspiration of fluid into the lung. Therefore, feeding rates into the small intestine are typically slow and may still be at risk for reflux.

A need therefore exists for an improved enteral safety feeding tube that allows for delivery of food and/or drugs at a higher flow rate while reducing the risk of reflux and/or aspiration.

### SUMMARY OF THE INVENTION

The enteral safe feeding catheter provided herein is useful for enteral rapidly delivery of drugs, food or any other material in solution into the stomach and small intestine. The enteral safe feeding catheter includes a safety system, which provides for automated monitoring and prevention of aspiration of fluids into the airway, in case of reflux from the stomach into the esophagus.

Accordingly, in one aspect, the enteral safe feeding catheter includes an inner tube having a first proximal end, a first distal end, and a first flexible wall surrounding an axis to define a primary lumen configured to deliver liquid food or drugs to a stomach or duodenum of a mammal, an outer tube having a second proximal end, a second distal end, and a second flexible wall surrounding the inner tube, a plurality of partition walls extending away from the axis and connecting an outer surface of the first flexible wall to an inner surface of the second flexible wall to define a plurality of secondary lumens surrounding the inner tube and configured to aspirate reflux fluid from the esophagus, and one or more environmental sensors disposed at the second distal ends of the one or more of the secondary lumens and configured to transmit a signal in response to a change in pH, pressure, temperature, oxygen, flow, moisture, or any combination thereof. The first distal end of the inner tube extends beyond the distal end of the outer tube and is configured for insertion into the stomach or duodenum of the mammal. The second distal end of the outer tube comprises a plurality of openings in fluid communication with each of the plurality of second lumens, and is configured to engage a cardia of the stomach of the mammal. The transmitted signal is configured to activate a suction pump connected to the one or more secondary lumens and to regulate the flow rate of an infusion pump connected to the primary lumen.

In various embodiments the catheter includes an inflatable balloon having a predetermined fill volume, disposed along the inner tube. In various embodiments, the catheter may include 2, 3, 4, 5, 6, 7, or 8 secondary lumens. In various embodiments, the enteral safe feeding catheter comprises at least one pH sensor disposed in the distal end of one of the secondary lumens. In such embodiments, the catheter may further include one or more of a pressure sensor, fluid flow sensor, or a temperature sensor. Each of the secondary lumens may be connected to a vacuum pump or peristaltic pump at the second distal end of the outer tube and the one or more environmental sensors and vacuum pump may be in electrical communication with an electronic microcontroller such that detection of a variation in pH, detection of fluid flow within the esophagus, detection of an increase in pressure within the stomach or esophagus, or any combination thereof causes the vacuum pump to aspirate reflux fluid from the esophagus and suspends flow of food or drugs within the inner tube. In various embodiments, the catheter may further include one or more optical fibers disposed within the first flexible wall and positioned along the axis, the optical fibers being configured to transmit light to a light emitter and imaging signals from an imaging sensor, wherein the light emitter and imaging sensor are disposed at the first distal end of the inner tube. In various embodiments, the catheter may further include a reporter disposed at the first distal end of the inner tube and configured to emit a detectable signal during insertion into a mammal. In various embodiments, the inner tube, outer tube, and partition walls are formed from medical grade polymer.

In another aspect, the invention provides an automated system for delivering liquid food or drugs to the stomach or duodenum of a mammal. The system includes the enteral safe feeding catheter described herein, a microcontroller in electrical communication with the one or more environmental sensors of the enteral safe feeding catheter, at least one first pump in electrical communication with the microcontroller and configured to deliver a liquid food or drug through the primary lumen of the enteral safe feeding catheter, and at least one second pump in electrical communication with the microcontroller and configured to generate negative pressure within the secondary lumens. The microcontroller may be configured to activate the at least one second pump in response to a signal received from the one or more environmental sensors.

In various embodiments, the microcontroller may be further configured to suspend the first pump in response to the signal received from the one or more environmental sensors. In various embodiments, the system also includes a microprocessor in electrical communication with the microcontroller, wherein the microprocessor is configured to analyze the signal received from the one or more environmental sensors and transmit a signal to the microcontroller to activate the at least one second pump, suspend the first pump, or both activate the at least one second pump and suspend the first pump. In various embodiments, the system also includes an alarm in electrical communication with the microprocessor and configured to alert medical personnel regarding the analyzed signals received from the environmental sensors or any changes performed by the microcontroller with regard to the first or second pumps.

In another aspect, the invention provides a method of rapidly delivering liquid food or drugs to the stomach or duodenum of a mammal. The method includes inserting the enteral safe feeding catheter described herein into the esophagus of the mammal, advancing the first distal end of the inner tube into the stomach or duodenum until the second distal end of the outer tube engages a cardia of the stomach of the mammal, and delivering liquid food or drugs to the stomach or duodenum through the primary lumen. In various embodiments, the method also includes monitoring one or more of pH, pressure, fluid flow, oxygen and moisture via environmental sensors disposed in the distal end of the one or more secondary lumens, wherein a change in pH, detection of fluid flow within the esophagus, detection of an increase in pressure within the stomach or esophagus, or any combination thereof activates a vacuum pump connected to the secondary lumens to aspirate reflux fluid from the esophagus and/or simultaneously suspends flow of food or drugs within the inner tube. As described herein, the electronic microcontroller may be configured to control the vacuum pump and to control delivery of the food or drugs flowing within the inner tube. In various embodiments, the step of inserting includes detecting an emitted signal from a reporter disposed at the first distal end of the inner tube to monitor to the position of the catheter while advancing into the esophagus of the mammal.

### BRIEF DESCRIPTION OF THE DRAWINGS

FIGS. 1A and 1B are pictorial diagrams showing an exemplary catheter inserted into the duodenum via the stomach and esophagus. FIG. 1B shows an enlargement of the portion of FIG. 1A that appears within a circled.

FIGS. 2A and 2B are pictorial diagrams showing an exemplary catheter inserted into the duodenum via the stomach and esophagus. FIG. 2B shows an enlargement of the portion of FIG. 2A that appears within a circled.

FIG. 3 is a pictorial diagram showing advancement of the catheter into the duodenum and inflation of a balloon at the pyloric valve.

FIG. 4 is a pictorial diagram showing a perspective view of a patient having an exemplary catheter inserted into its esophagus.

FIG. 5 is an exemplary system for automated delivery of liquid food or drugs to a mammal.

### DETAILED DESCRIPTION OF THE INVENTION

The enteral safe feeding catheter provided herein is useful for enteral delivery of drugs, food or any other material in solution into the stomach and small intestine. The enteral safe feeding catheter includes a safety system, which prevents aspiration of fluids into the airways, in case of reflux from the stomach into the esophagus.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The enteral safe feeding catheter provided herein is useful for enteral delivery of drugs, food or any other material in solution into the stomach and small intestine. The enteral safe feeding catheter includes a safety system, which prevents aspiration of fluids into the airways, in case of reflux from the stomach into the esophagus.

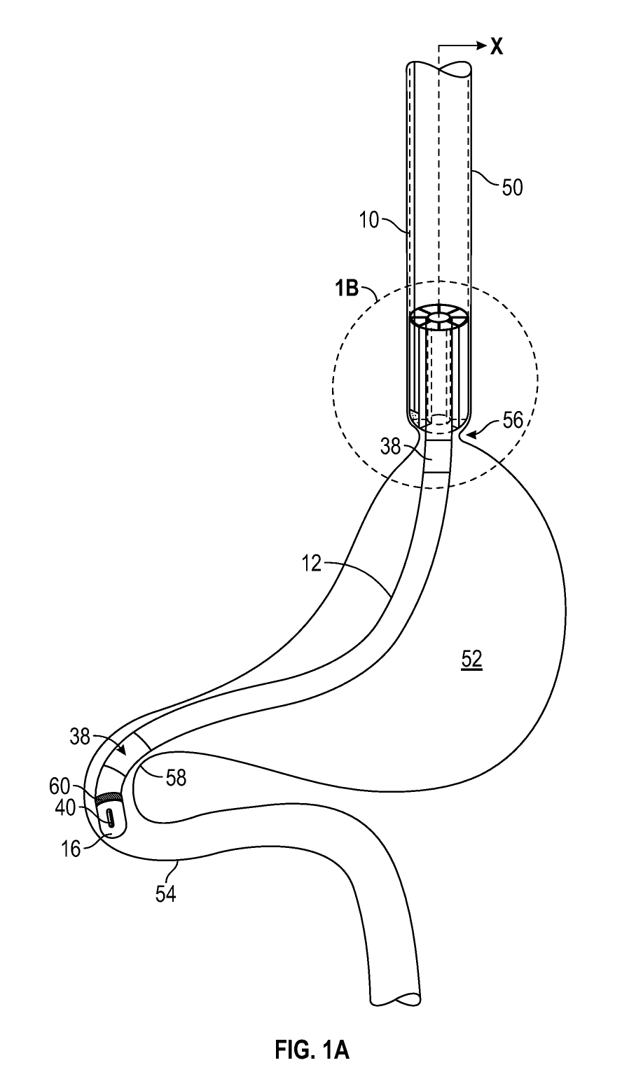

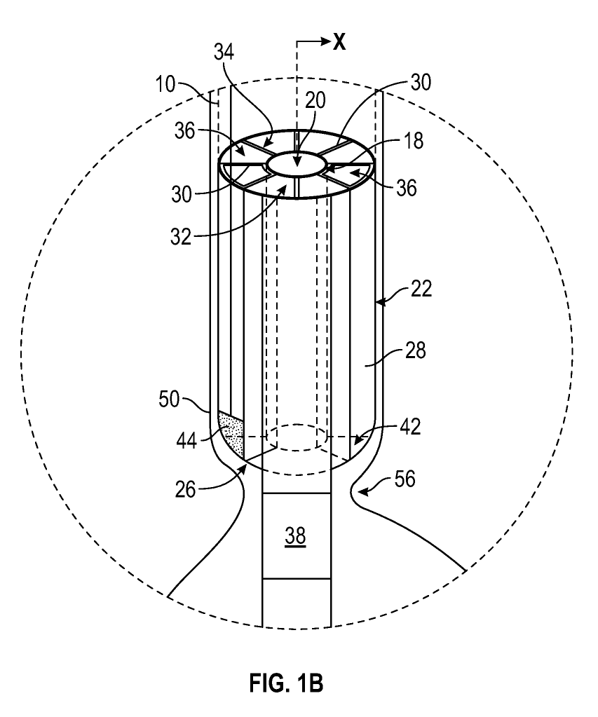

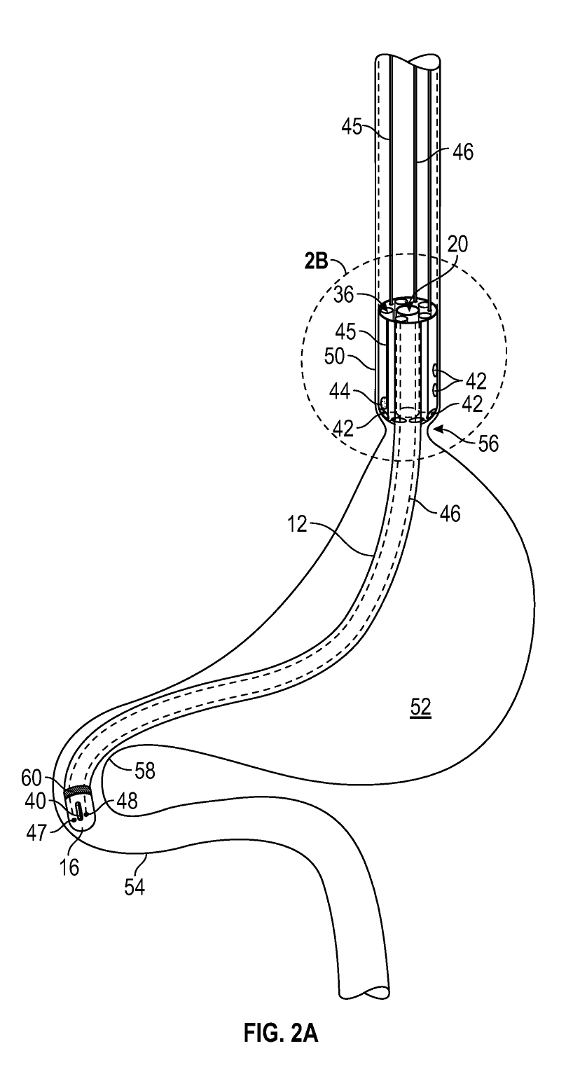

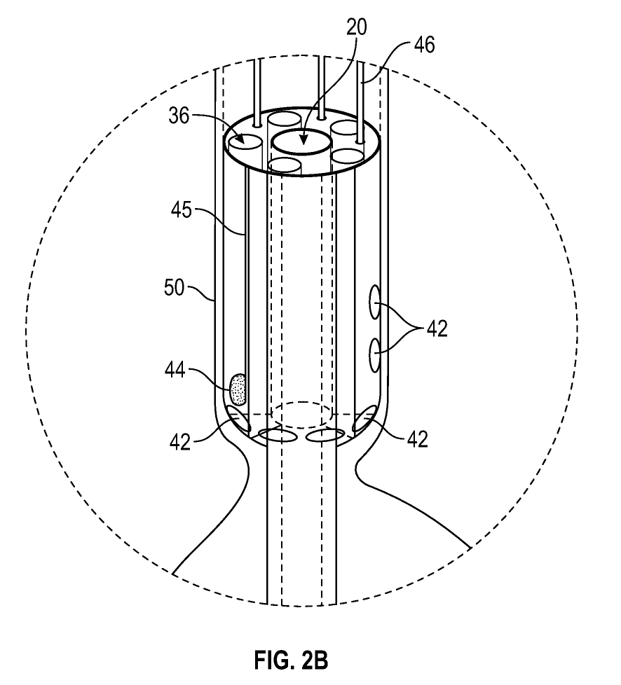

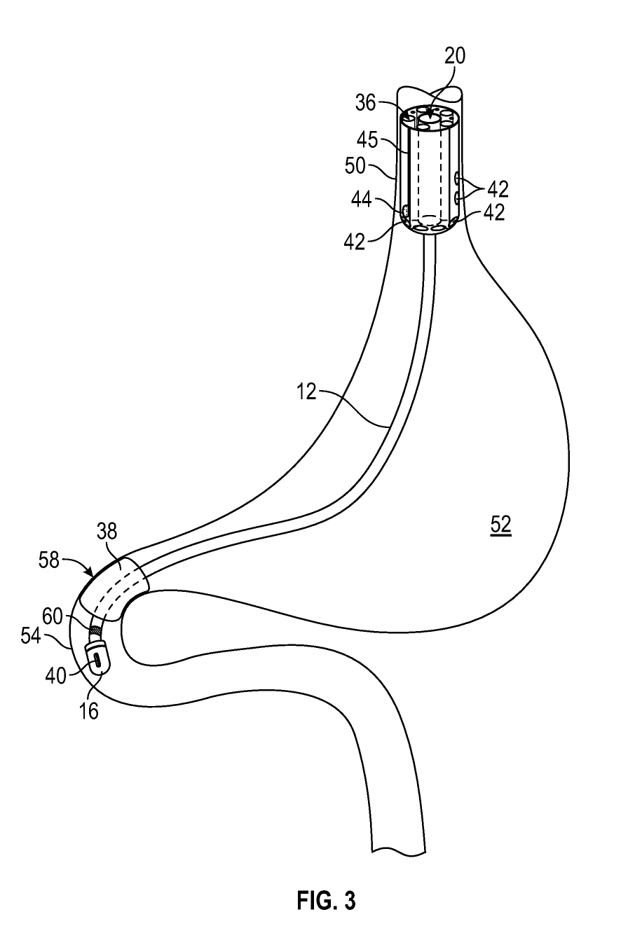

Referring now to FIGS. 1 and 2, in one aspect, the enteral safe feeding catheter 10 includes an inner tube 12 having a proximal end 14, a first distal end 16, and a first flexible wall 18 surrounding an axis X to define a primary lumen 20 configured to deliver liquid food or drugs to a stomach or duodenum of a mammal, an outer tube 22 having a second proximal end 24, a second distal end 26, and a second flexible wall 28 surrounding the inner tube 12, and a plurality of partition walls 30 extending away from the axis X and connecting an outer surface 32 of the first flexible wall 18 to an inner surface 34 of the second flexible wall 28 to define a plurality of secondary lumens 36 surrounding the inner lumen 20 and configured to aspirate reflux fluid from the esophagus 50 or stomach 52. The first distal end 16 of the inner tube 12 extends beyond the second distal end 26 of the outer tube 22 and is configured for insertion into the stomach 52 or duodenum 54 of the mammal. The second distal end of the outer tube comprises a plurality of openings in fluid communication with each of the plurality of second lumens, and is configured to engage a cardia of the stomach of the mammal. In certain embodiments, an inflatable balloon 38 having a predetermined fill volume may be disposed within the first flexible wall 18 at a location along the inner tube 12 that corresponds to the cardia 56, the pyloric valve 58, or both the cardia 56 and the pyloric valve 58 when the catheter 10 is inserted into the esophagus 50 of the mammal. Provided at the first distal end 16 is an opening 40 in fluid communication with the inner lumen 20, and configured to deliver liquid food or drugs into the stomach 52 or duodenum 54 of the mammal (see FIG. 3).

In various embodiments, the inner lumen 20 and the secondary lumens 36 may be generally cylindrical in shape. However, any shape or combination of shapes may be used in the catheter 10. Likewise, the number of secondary lumens 36 may range from 2 to 10 (i.e., 2, 3, 4, 5, 6, 7, 8, 9, or 10), depending on the needs of the user. Exemplary multilumen tubing of various sizes, shapes, and configurations that may be useful in the catheter 10 of the present invention are commercially available from Enki Engineering & Manufacturing (Concesio, Brescia, Italy).

The design of the enteral safety feeding tube 10 allows the multitude of surrounding secondary lumens 36 to be connected (either collectively or independently) to one or more vacuum pumps for aspiration of reflux material from the stomach 52 or esophagus 50, thereby avoiding aspiration of the fluid into the lungs. Thus, in various embodiments, the enteral safety feeding tube 10 may include two, three, four, five, six, seven or eight secondary lumens 36. In certain embodiments, the safety feeding tube 10 includes six or eight secondary lumens 36, each at the same initial pressure, and each having one or more openings 42 in fluid communication with each of the plurality of second lumens 36. Thus, in various embodiments, each secondary lumen 36 may have one or more (i.e., 1, 2, 3, 4, 5, 6, 7, 8 or more) corresponding openings 42.

Without being bound by theory, the rationale of this design is that: i) having several secondary lumens 36 surrounding the inner tube 12 (i.e., primary lumen 20) reduces the risk of clogging the vacuum line; ii) having a plurality of openings 42 corresponding to each of the secondary lumens 36 will enhance the protection of the upper airways by maximizing the collection of reflux, while minimizing negative pressure necessary to apply within the safety tube 10; iii) the plurality of openings 42 for each secondary lumen 36 likewise reduces the risk of suction of the esophageal wall; and iv) the plurality of openings 42 maximizes the ability to aspirate any regurgitated flow in each possible direction of flow around the outer tube 22.

In various embodiments, the enteral safe feeding catheter 10 may include one or more environmental sensors 44 disposed therein to detect environmental changes within the esophagus 50 and/or stomach 52 of the subject being treated. In various embodiments, the environmental sensors 44 may be embedded within the second flexible wall 28 of the outer tube 22, embedded within any of the partition walls 30, embedded within the first flexible wall 18 of the inner tube 12, or any combination thereof. Alternatively, or in addition thereto, the environmental sensors 44 may be fixedly mounted (e.g., with adhesive or other means) to any surface of the above-mentioned walls, so long as the environmental sensory 44 is able to sense the parameter for which it is designed. In various embodiments, the environmental sensors may be disposed at one or more openings 42 of the secondary lumens 36. Signals from each of the environmental sensors 44 may be transmitted along one or more wires 45, which may be disposed within the catheter 10 along axis X. Such wires 45 may extend beyond the proximal end of the catheter 10 to provide electrical communication with a microcontroller 110, as described in more detail below.

Exemplary environmental sensors include, but are not limited to, pH sensors, pressure sensors, fluid flow sensors, temperature sensors, and moisture sensors, each of which being configured to independently monitor and/or detect changes in pH, pressure, fluid flow, temperature, oxygen content, and/or moisture within the esophagus 50 and/or stomach 52. In certain embodiments, one of the six or eight secondary lumens 36 will be equipped with a pH sensor configured for detection of reflux using established technologies in the field of gastroesophageal reflux detection. For example, a secondary lumen 36 may be equipped with an antimony or a glass electrode configured to detect pH. Since the average pH within the esophagus 50 is about 7 and the average pH within the stomach is about 2, any variation of pH serves to indicate a possible reflux from the stomach 52 into the esophagus 50. Any change in detected pH may be used to activate a vacuum pump in order to ensure that no reflux fluid reaches the level of the trachea, thereby preventing potential aspiration into the airway. In certain embodiments, detection of an environmental change will simultaneously suspend delivery of the liquid food and/or drug flowing within the primary lumen 20.

The secondary lumens are therefore configured to drain the reflux fluid through the openings 42, driven by gravitational pressure, a vacuum pump, or both in succession. In case of regurgitation, the gravitational gradient enables suction of the backward flow through the openings 42; at the same time, the one or more secondary lumens 36 that contain the environmental sensors 44 (e.g., a pH sensor, pressure sensor, or fluid flow sensor) in close proximity to the corresponding openings 42, which is located at or near the cardia 56 and/or the lower esophagus 50 when the catheter 10 is inserted into a mammal, may be used to activate a vacuum pump and begin aspiration of the regurgitated fluid. In addition thereto, the signals generated by the environmental sensors 44 may further stop the infusion line outflow if necessary.

In various embodiments, the catheter 10 may further include one or more optical fibers 46 disposed within the first flexible wall 18 of inner tube 12. Each optical fiber 46 may have a first end disposed at or in close proximity to the first distal end 16 of the inner tube 12 (i.e., near the opening 40), and a second end configured for optical communication with a light source and/or and imaging device.

As used herein, an “optical fiber” refers to a flexible, transparent fiber made of glass or plastic that functions as a waveguide to transmit light between the two ends (i.e., a first end and a second end) of the fiber. Typically, optical fibers include a transparent core surrounded by an opaque cladding material with a lower index of refraction and low to no autofluorescence characteristics. It should be understood that an optical pathway or assembly comprising the optical fiber may optionally include one or more filters, lenses, aspheres, etc., to modify and/or focus emission signals and imaging signals passing therethrough. Thus, the catheter may be provided with at least one imaging sensor 47, such as a CCD and an objective lens, and at least one light emitter 48 disposed at or in close proximity to the first distal end 16 of the inner tube 12. Exemplary miniaturized cameras useful in the catheter 10 of the present invention are described in U.S. Pub. No. 2014/0005480, and U.S. Pat. Nos. 5,016,098 and 5,667,478, each of which is hereby incorporated by reference in their entireties.

In various embodiments, the catheter 10 is flexible, which allows for easy insertion in the esophagus 50 through the mouth or nose, and facilitates placement in the duodenum 54. As described above, the distal end 26 of the outer tube 22 may be configured to engage the cardia 56 of the stomach. Thus, the catheter 10 may be inserted as a nasopharyngeal or oral tube along the esophagus 50 into the stomach 52 and moved through the pylorus valve 58 into the duodenum 54, for final placement. In various embodiments, the tip 16 can be placed in the stomach 52 or duodenum 54, according to the desired application.

In various embodiments, the catheter 10 may further include a reporter 60 disposed at or in close proximity to the first distal end 16 of the inner tube 12 (i.e., near the opening 40). The reporter 60 may embedded within the material forming the inner tube 12 or may be adhered thereto, and configured to generate a detectable signal for guiding the catheter 10 during placement. In various embodiments the report 60 may be a magnetic energy generator or magnetic field generator for used in conjunction with known signal generating placement control devices/sensors/systems, such as those available from Corpak Medsystems, Inc. (Wheeling, Ill.); see, e.g., US Pat. Nos. 7,976,518; 8,197,494; 8,265,732; 8,606,347; 8,606,347; 8,934,960; 9,028,441; and 9,131,956, all of which are incorporated herein by reference in their entireties. In various embodiments, the report 60 may include a radio isotope or fluorophore to aid in detectable guidance of insertion of the catheter 10.

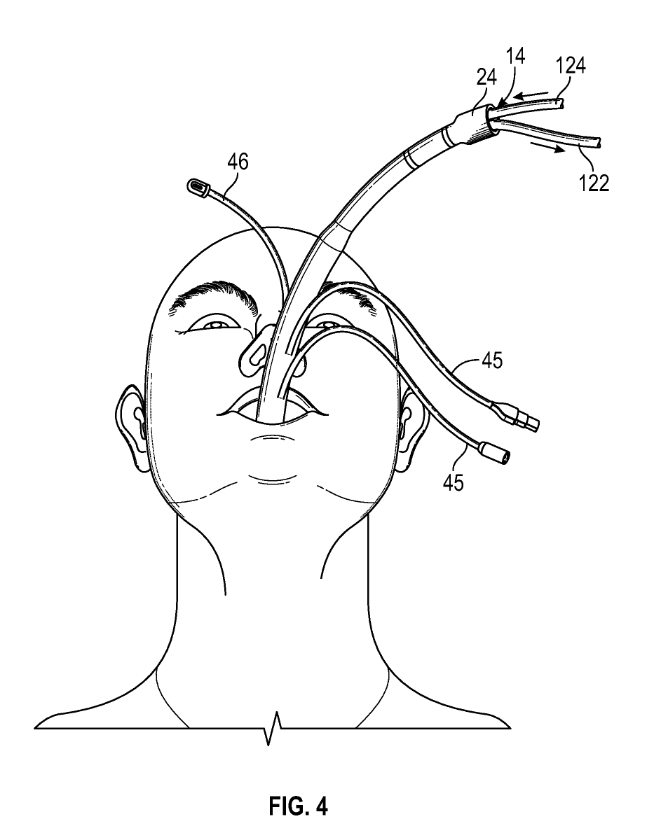

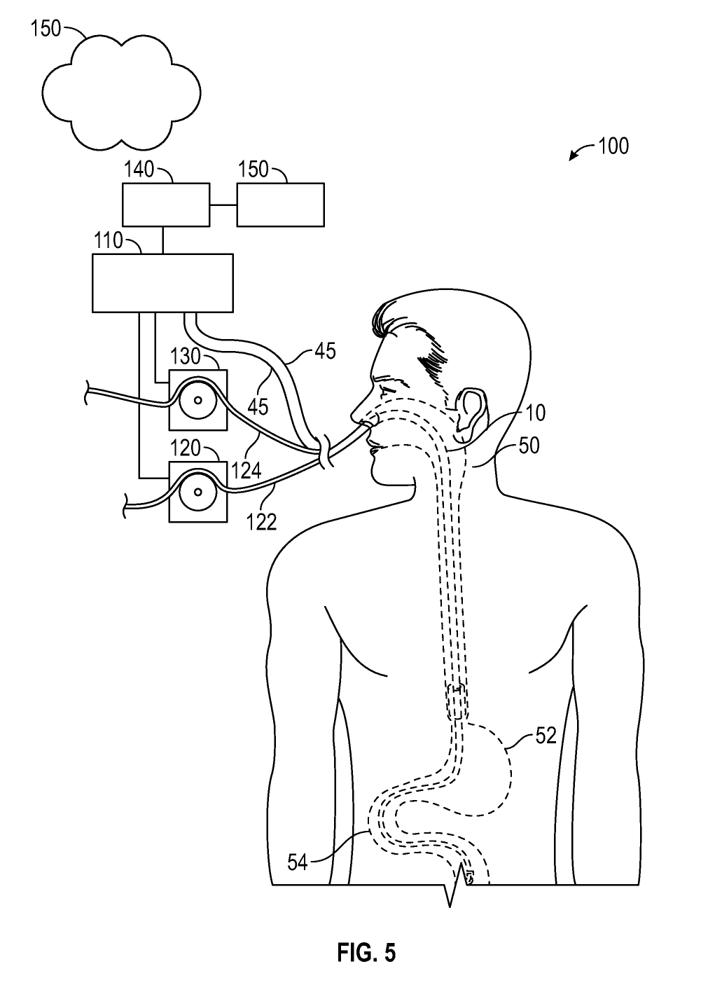

With reference now to FIGS. 4 and 5, in another aspect, the invention provides an automated system 100 for monitored feeding and/or infusion of a liquid drug. The system includes the catheter 10, as described above, an electronic controller 110 that is in electrical communication with the one or more environmental sensors 44, and at least one pump 120 in electrical communication with the electronic controller 110. In various embodiments, the at least one pump 120 may be a single or multichannel peristaltic pump to which tubing 122 corresponding to each of the surrounding lumens 36 to be used for removal of reflux is attached. Alternatively, the at least one pump 120 may be a vacuum pump configured to generate a negative pressure within the esophagus 50 and aspirate any reflux therein. In various embodiments, the system 100 may include a second pump 130 in electrical communication with the electronic controller 110. The second pump 130 may also be a peristaltic pump to which tubing 124 corresponding to the primary lumen 20 for infusion of the liquid food or drug is attached. While pumps 120 and 130 are shown as separate units, it should be understood that a single multichannel pump may be used in place of pump 120 and pump 130, provided that the multichannel pump is configured for independent control of each channel thereof. Electronic controller 110 may further be configured for electrical communication with the one or more environmental sensors 44. As such, electronic controller 110 may receive signals from each of the environmental sensors 44, and in response thereto, generate signals to turn on, turn off, and/or reverse direction of each of the first pump 120 and second pump 130.

Accordingly, in response to, for example, a change in esophageal pH (which would be indicative of a reflux situation), the microcontroller 110 may activate the vacuum pump 120 to initiate suction through openings 42. Negative pressure increases within each of the secondary lumens 36, thereby enabling safe aspiration of all regurgitation fluid out of esophagus 50. Depending on the treatment protocol, the microcontroller may further suspend the flow of infusion solution through the primary lumen 20 until the regurgitation fluid has been removed from the esophagus 50.

Alternatively, or in addition thereto, detection of fluid flow within the esophagus 50 where fluid flow is not expected, would be indicative of a backward flow from the stomach 52 into the esophagus 50. Since such reflux of fluid into the esophagus 50 may result in aspiration of the fluid into the airway of the patient, detection of fluid flow within the esophagus by a fluid flow sensor (i.e., environmental sensor 44), may trigger the microcontroller 110 to activate the vacuum pump 120 to initiate suction through openings 42, thereby increasing negative pressure within each of the secondary lumens 36 and enabling safe aspiration of all regurgitation fluid out of esophagus 50. Depending on the treatment protocol, the microcontroller may further suspend the flow of infusion solution through the primary lumen 20 until the regurgitation fluid has been removed from the esophagus 50.

Alternatively, or in addition thereto, detection of an increase in pressure within the esophagus 50 and/or stomach 52 beyond a predetermined threshold would be indicative of a backward flow from the stomach 52 into the esophagus 50. Again, since an increase in pressure may result in an increased risk for backward fluid flow into the esophagus 50, and ultimately into the airway of the patient, the detected increase by a pressure sensor (i.e., environmental sensor 44) may trigger the microcontroller 110 to activate the vacuum pump 120 to initiate suction through openings 42, thereby increasing negative pressure within each of the secondary lumens 36 and enabling safe aspiration of all regurgitation fluid out of esophagus 50. Depending on the treatment protocol, the microcontroller may further suspend the flow of infusion solution through the primary lumen 20 until the regurgitation fluid has been removed from the esophagus 50.

In various embodiments microcontroller 110 may include a microprocessor 140 in electrical communication with the microcontroller 110, and configured to analyze signals received from the environmental sensors 44 and determine the most appropriate actions with regard to infusion and/or aspiration. In certain embodiments, microprocessor 140 may be separate unit from microcontroller 110, and may further be connected to an alarm 150 configured to alert medical personnel of the analyzed signals received from the environmental sensors 44 and/or any changes made by the microcontroller 110 to the direction and/or speed of the first pump 120 and/or second pump 130. Alarm 150 may include an independent computer located at, for example, a nurse's station or may be a wireless laptop or tablet computer carried by medical professionals for real-time monitoring of the subject being treated. Microprocessor 140 may further be configured to connect to a signal generating device for sensing the position of reporter 60 and/or may include a light source for transmission through the optical fiber 46 to the light emitter 48 and/or may be configured to receive imaging signals from the imaging sensor 47.

While system 100 is shown as incorporating wired connectivity for the electrical communication amongst the components, it should be understood that one or more of the communication connections may be accomplished via one or more wireless communication protocols. The wireless communication may be selected from the group consisting of infrared transmission, Bluetooth protocol, radio frequency, Zigbee wireless technology, GPS, Wi-Fi, WiMAX, and mobile telephony, and may be configured to send/receive signals generated by the environmental sensors 44 and/or to the first pump 120 and/or to the second pump 130 and/or to the alarm 150.

Early pilot tests of the catheter 10 included use of a syringe pump for the delivery line (i.e., primary lumen 20), and a syringe providing negative pressure for the safety suction line (i.e., secondary lumens 36). In addition, a fluid filled catheter for the continuous, high fidelity monitoring of intragastric pressure, has been tied to the delivery catheter. More recent tests have been carried out with a multichannel peristaltic pump with independent control of the outflow and vacuum lines (i.e., delivery and safety tubes), as described in more detail below.

Thus, it is envisioned that the device provided herein may be useful in a treatment regimen for shock. Recent evidence suggests that a major component of inflammatory mediators from the intestine in shock cause multi-organ failure and mortality (e.g., after surgery/general anesthesia, trauma, chronic diseases and any other condition leading multi-organ failure) is derived from the action of pancreatic lipases (lipid splitting enzymes). Blockade of pancreatic lipase serves to reduce mortality during shock and reduce inflammation that leads to multi-organ failure. Blockade of pancreatic lipase prior to general anesthesia may serve to preserve barrier properties of the intestinal mucosa, reduce inflammation in the central circulation, and consequently reduce recovery and wound healing periods, post-surgical complications, hospital stays, etc.

The inventors have previously shown that an ischemic intestine produces a powerful set of lipid-derived cytotoxic mediators, and that the blockade of lipase in the intestine under in vitro conditions blocks the production of lipid-derived cytotoxic mediators (see, e.g., U.S. Pat. Nos. 6,534,283; 8,541,371; and 9,272,034; and US Pub. Nos. 20130231309; 20130281660; 20150010644; 20150297619; 20160144007; and 20160367642, the content of each of which is incorporated herein by reference). In elective surgery, pre-administration of a pancreatic enzyme inhibitor directly into the lumen of the intestine (by oral administration, introduction via an esophageal catheter, direct injection into the lumen of the intestine during surgery) may therefore have a positive effect on recovery. The agents to be used are individually or in combination: orlistat (5 to 50 mg/ml), lipase inhibitor; tranexamic acid (127 mM) plus any other pancreatic enzyme inhibitor. The amount administered is adjusted according to intestine size and content to achieve complete blockade of digestive enzyme activity. As treatment, the inhibitor may be administered after trauma or sepsis associated with risk for shock and multi-organ failure. As pretreatment, the inhibitor may be administered prior to general anesthesia/surgery.

Thus, the catheter 10 and system 100 may be used to rapidly administer a composition that blocks lipase and protease activity in the lumen of the intestine to maintain the mucosal barrier. In cases in which lipase and/or protease escapes from the lumen of the intestine into the circulation, such treatment may prevent formation of lipid-derived inflammatory or cytotoxic mediators and of signaling or inflammatory mediator peptides in shock and other inflammatory diseases and attenuate multi-organ failure in shock and chronic inflammation in such diseases as hypertension, diabetes, the metabolic syndrome, cancers and in chronic degenerative diseases.

The following examples are intended to illustrate but not limit the invention.

### EXAMPLE 1

The system was initially tested in Wistar rats (weighing 400 gm-660 gm) to deliver a solution containing a protease inhibitor into the intestine during hemorrhagic shock experiments. Previous studies suggest that the optimal placement of the catheter for the purpose of delivering the drug to the small intestine is within the duodenum. Placement of the catheter in the stomach may result in the inflation of the stomach because the stomach emptying rate was observed to be slower than the infusion rate. Therefore, the indication is to place the tube in the duodenum in order to reduce the risk for reflux when administering large volumes of solution at high rate.

Brief Summary—A ketamine/xylazine anesthesia cocktail was administered to the rats (0.79 mg/kg). Hemorrhagic shock was induced via blood withdrawal through the venous line, with a target basal intra-gastric pressure of about 35 mmHg. After maintaining hypovolemia for two hours, all the shed blood was returned. If a sample of the blood was collected for measurements such as, for example, blood lactate, creatinine, glucose, blood gas analysis, and for omics analyses, an equal volume of Ringer lactate was infused with the rest of the blood initially shed to reach the target pressure. Duration of reperfusion was about two hours.

Enteral delivery of tranexamic acid (Cyklokapron)—Vehicle solution: GoLytely in sterile water (0.068 g/ml); 0.25 ml Cyklokapron/1 ml of GoLytely solution. A dual-lumen catheter (i.e., for demonstration purposes only, two tubes were tied together with one being the “infusion tube”, and the other being the “safety tube”) was connected to a multichannel peristaltic pump and inserted in the mouth at ˜10 minutes into hypovolemia (after the animal has fully transitioned into the compensatory phase following bleeding). Before inserting the tube into the mouth, the infusion pump was started (infusion line on the multichannel pump) to prime the infusion tube and avoid pumping air into the stomach and intestine of the animal.

A laparotomy was carried out for manual placement of the tip of the infusion tube into the duodenum (about 1 inch after the pyloric sphincter). The way the two tubes are tied together ensures that the tip of the safety tube is placed in the lower esophagus. The pump on the safety tube was started as soon as the system was in place, at a negative pressure identical in absolute value to the infusion pressure.

After several tests at different pumping rates, the rate considered optimal (i.e., a tradeoff between minimizing the time of infusion to achieve filling of the intestine in the shortest possible time and preventing inflation of the stomach and an induction of reflux into the esophagus) in rats is ˜0.11 ml/min. The total volume infused into the intestine of the rats was 17.5 ml; total duration of infusion was 2 hours 30 minutes. At this infusion rate, reflux was not observed; however, the safety system was able to intercept some backward flow in the presence of higher infusion rates (up to ˜0.25 ml/min), thereby preventing aspiration into the airway.

A total of eleven rat experiments were performed (six having intragastric infusion and five having duodenal infusion). Performance in terms of filling of the small intestine was consistently >90% of the total length of the small intestine (verified at euthanasia). In one case, filling of the cecum was also observed.

### EXAMPLE 2

12 male Wistar rats were randomly assigned to two experimental hemorrhagic shock groups: (i) Control: enteral infusion of vehicle (GoLytely); and (ii) Treatment: enteral infusion of a protease inhibitor (Tranexamic Acid, 127 mM) in GoLytely solution (0.068 g/ml GoLytely in sterile water). The infusion line was placed in the duodenum post-pyloric, while the safety lines were placed intragastric or esophageal.

Hemorrhagic shock was induced by sedation followed by blood withdrawal to a target pressure of about 35 mmHg. The infusion line was inserted at about 10 minutes after completion of hemorrhage and infusion of about 17.5 cc of solution progressed over 2.5 hours. After two hours of ischemia, the removed blood was returned followed by two hours of reperfusion.

All major endpoints were improved/preserved in shock with the proposed treatment (arterial pressure, lactate, blood gas, etc.) Histology showed improved preservation and integrity of the intestinal barrier morphology. Thus, demonstrating that a continuous enteral infusion of protease inhibitor is a viable treatment of an acutely ill animal in circulatory shock.

### EXAMPLE 3

The system was subsequently tested in pigs for delivery of a solution into the intestine to simulate rapid infusion treatment of a subject during hemorrhagic shock. The pig experiments were conducted on four separate occasions. In all experiments, female pigs (weighing approximately 40 kg) were anesthetized with propofol or a combination of propofol and midazolam. In some cases, anesthesia was initially induced by intramuscular injection of ketamine. Pigs were usually intubated and mechanically ventilated. The duration of the experiments ranged from about 2.5 hours to 11 hours.

The solutions administered were Ringer lactate+barium (first experiment); water+GoLytely solution+food coloring (second experiment); water+GoLytely solution+contrast and methylene blue (third experiment); and water+barium (fourth experiment).

Standard nasogastric tubes (18 Fr or smaller Salem tubes) were used in all pig experiments. There was no problem with the placement of two tubes in the esophagus, even in the presence of an endotracheal tube (an endotracheal tube was used in second and fourth experiments only). A variety of pumps (e.g., intravenous infusion pump, peristaltic pump, and syringe pump) were tested for efficacy. In the first and fourth experiments, a calibrated peristaltic pump was used, with a flow of ˜20 ml/min with the goal of delivering about 2 liters of solution to the intestine. Furthermore, it was determined that insertion of the infusion tube into the duodenum blindly was difficult. As such, gastroscopy was used in order to advance the tube beyond the pyloric valve in some experiments.

In the first experiment, about two liters were delivered within about 40 minutes. Despite the placement of the infusion tube in the stomach, there was no apparent inflation of the stomach and the fluoroscopic images showed an efficient distribution of the infused fluid along the small intestine.

In the second experiment, about 750 ml of solution was delivered in about 10 minutes. This procedure was repeated three times, at intervals of about 30 minutes. The stomach appeared to be considerably inflated after the first infusion, but this did not prevent the fluid from advancing into the intestine. Without being bound by theory, it is possible that the effect of inflation of the stomach was magnified by the fact that this animal was kept with the peritoneum open for the duration of the experiment (a loose suture was applied externally to keep the abdominal cavity not exposed to atmospheric air). Fluoroscopic images showed that the infused fluid advanced into the small intestine and reached the distal portion thereof. After sacrifice, it was observed that some infusion fluid reached the large intestine. Even though the animal was fasted overnight, there was noticeable food residue in the small intestine. However, the food residue did not constitute an obstacle to the advancement of the fluid into the intestine.

In the third experiment, about two liters of solution was delivered at a fixed rate of 20 ml/min. Due to a misplacement of the infusion tube in the stomach, there was initially reflux, which was at least in part collected by the safety tube. Both fluoroscopic images and visual inspection of the abdomen at the end of the procedure showed little diffusion of the solution along the intestine. Without being bound by theory, it was observed that a misplacement of the tip of the infusion catheter in the stomach (i.e., too close to the cardia) induced reflux at the beginning of infusion (this was observed in the rat experiments as well); and filling of the stomach and distension of its walls (observed both in rats and pigs when the catheter is in the stomach) contributes to reflux.

In the fourth experiment, about 1.3 liters of solution was delivered at a fixed rate of 10 ml/min. Before insertion of the catheter for infusion of the solution, the stomach was rinsed with a gastroscope by flushing it twice via infusion of 500 ml of water, which was then drained out of the stomach together with possible fluid and solid residuals still present despite the pre-operative fasting of the animal.

After about two hours of infusion, it was observed that the small intestine was filled, there were traces of fluid in the large intestine, and no sign of reflux into the stomach. However, post-mortem tests involving doubling the infusion rate showed that a faster infusion rate may also facilitate backward flow from the duodenum into the stomach. Intragastric pressure was monitored during the whole duration of the infusion process. Consistent with the post-mortem observation that no fluid flowed back from the duodenum into the stomach, intragastric pressure remained stable throughout the experiment.

The infusion rate was therefore optimized to minimize the risk of backward flow into the stomach, while maximizing the infusion of the solution into the small intestine. It was observed that a rate of about 0.10 to 0.15 ml/min in rats and about 20 ml/min in swine was achievable while preventing backflow into the stomach. In both rats and pigs, very efficient filling of the small intestine was achieved (e.g., 90% of the small intestine in rats was filled on average after delivering 15 to 20 cc over 2 to 3 hours), while reflux flow was not observed during the recommended infusion rate parameters and catheter placement protocol.

### EXAMPLE 4

It was decided to test the possibility of operating the safety system inside the stomach rather than the esophagus, according to the same principles validated in rats. In this experiment, the “safety line” was connected to an aspiration pump that continuously generated a low level of negative pressure to ensure both that the aspiration of possible backward flow was continuously performed and that there was no risk of suction of the esophageal/gastric walls.

A comparison of the efficiency of suction in the stomach vs. esophagus was performed in vitro. A simple control system using infusion pumps driven by pressure measurements obtained from a pressure sensor within the stomach was prepared.

### EXAMPLE 5

For validation, the results obtained by administration of the protease inhibitor via EnterSafe/EnterSafeCath were compared to those obtained via the non-translational approach based on multiple enteral injections. It was observed that: a) protease treated animals displayed a stable blood pressure in reperfusion following shock with both delivery methods; b) the morphology of the intestine is better preserved with the treatment with both delivery methods; c) some blood biochemical parameters are improved in the treated animals. Thus, the present invention makes possible the flushing of the intestine for the delivery of specific enzyme inhibitors in trauma and circulatory shock patients with minimal peristaltic motion in their gastrointestinal track. For this reason, it is likely that the device will be of interest more in general in the field of enteral drug delivery.

### EXAMPLE 6

For further validation, a commercially available multilumen catheter for duodenal manometry was obtained for in vitro testing and further animal experiments (pigs). The catheter consisted of nine lumens (one central lumen with eight surrounding lumens). The central lumen was used for infusion, while some of the surrounding lumens were used for suction. Other surrounding lumens were used for environmental sensors, such as, for example, intragastric pressure measurement via connection to a pressure transducer.

The catheter was connected to a two-channel Ismatec Reglo ICC pump and to the in vitro system simulating access to the duodenum through the pyloric valve. Testing of the catheter showed the ability to deliver the flow rates required for swine infusion, and that the surrounding aspiration lines were able to function correctly.

Although the invention has been described with reference to the above examples, it will be understood that modifications and variations are encompassed within the spirit and scope of the invention. Accordingly, the invention is limited only by the following claims.

1. An enteral safe feeding catheter comprising:
(a) an inner tube having a first proximal end, a first distal end, and a first flexible wall surrounding an axis to define a primary lumen configured to deliver liquid food or drugs to a stomach or duodenum of a mammal;
(b) an outer tube having a second proximal end, a second distal end, and a second flexible wall surrounding the inner tube;
(c) a plurality of partition walls extending away from the axis and connecting an outer surface of the first flexible wall to an inner surface of the second flexible wall to define a plurality of secondary lumens surrounding the inner tube and configured to aspirate reflux fluid from the esophagus; and
(d) one or more environmental sensors disposed at the second distal ends of the one or more of the secondary lumens and configured to transmit a signal in response to a change in pH, pressure, flow, temperature, oxygen, moisture, or any combination thereof,
wherein the first distal end of the inner tube extends beyond the distal end of the outer tube and is configured for insertion into the stomach or duodenum of the mammal,
wherein the second distal end of the outer tube comprises a plurality of openings in fluid communication with each of the plurality of second lumens, and is configured to engage a cardia of the stomach of the mammal, and
wherein the transmitted signal is configured to activate a suction pump connected to the one or more secondary lumens and to regulate the flow rate of an infusion pump connected to the primary lumen.

2. The enteral safe feeding catheter of claim 1, further comprising at least one inflatable balloon having a predetermined fill volume, disposed along the inner tube.

3. (canceled)

4. The enteral safe feeding catheter of claim 1, wherein the one or more environmental sensors are independently selected from the group consisting of pH sensor, pressure sensor, flow sensor, oxygen sensor, temperature sensor, and moisture sensor.

5. (canceled)

6. The enteral safe feeding catheter of claim 1, wherein each of the secondary lumens is connected to a vacuum pump or peristaltic pump at the second distal end of the outer tube.

7. The enteral safe feeding catheter of claim 1, wherein each of the secondary lumens is connected to a vacuum pump at the second distal end of the outer tube, and wherein the one or more environmental sensors and vacuum pump are in electrical communication with an electronic microcontroller such that detection of a variation in pH causes the vacuum pump to aspirate reflux fluid from the esophagus and suspends flow of food or drugs within the inner tube.

8. (canceled)

9. The enteral safe feeding catheter of claim 7, further comprising a fluid flow sensor in electrical communication with the electronic microcontroller, wherein detection of fluid flow within the esophagus causes the microcontroller to activate the vacuum pump to aspirate reflux fluid from the esophagus and to suspend flow of food or drugs within the inner tube.

10. (canceled)

11. The enteral safe feeding catheter of claim 9, further comprising a pressure sensor in electrical communication with the electronic microcontroller, wherein detection of an increase in pressure within the stomach or esophagus causes the microcontroller to activate the vacuum pump to aspirate reflux fluid from the esophagus and to suspend flow of food or drugs within the inner tube.

12. (canceled)

13. The enteral safe feeding catheter of claim 1, wherein each of the secondary lumens is connected to a vacuum pump at the second distal end of the outer tube, and wherein the one or more environmental sensors and vacuum pump are in electrical communication with an electronic microcontroller such that detection of fluid flow within the esophagus or detection of an increase in pressure within the stomach or esophagus causes the vacuum pump to aspirate reflux fluid from the esophagus and suspends flow of food or drugs within the inner tube.

14-16. (canceled)

17. The enteral safe feeding catheter of claim 1, further comprising one or more optical fibers disposed within the first flexible wall and positioned along the axis, the optical fibers being configured to transmit light to a light emitter and imaging signals from an imaging sensor, wherein the light emitter and imaging sensor are disposed at the first distal end of the inner tube.

18. The enteral safe feeding catheter of claim 1, further comprising a reporter disposed at the first distal end of the inner tube and configured to emit a detectable signal during insertion into a mammal.

19-21. (canceled)

22. The enteral safe feeding catheter of claim 1, wherein the second distal end of the outer tube comprises more than one opening per secondary lumen.

23. The enteral safe feeding catheter of claim 4, wherein the pH is an antimony or glass electrode.

24. An automated system for delivering liquid food or drugs to the stomach or duodenum of a mammal comprising:
(a) the enteral safe feeding catheter of claim 1;
(b) a microcontroller in electrical communication with the one or more environmental sensors of the enteral safe feeding catheter;
(c) at least one first pump in electrical communication with the microcontroller and configured to deliver a liquid food or drug through the primary lumen of the enteral safe feeding catheter; and
(d) at least one second pump in electrical communication with the microcontroller and configured to generate negative pressure within the secondary lumens,
wherein the microcontroller is configured to activate the at least one second pump in response to a signal received from the one or more environmental sensors.

25. The automated system of claim 24, wherein the microcontroller is further configured to suspend the first pump in response to the signal received from the one or more environmental sensors.

26. The automated system of claim 24, further comprising a microprocessor in electrical communication with the microcontroller, wherein the microprocessor is configured to analyze the signal received from the one or more environmental sensors and transmit a signal to the microcontroller to activate the at least one second pump, suspend the first pump, or both activate the at least one second pump and suspend the first pump.

27. The automated system of claim 26, wherein the one or more environmental sensors are independently selected from the group consisting of pH sensor, pressure sensor, flow sensor, oxygen sensor, temperature sensor, and moisture sensor.

28. The automated system of claim 26, further comprising an alarm in electrical communication with the microprocessor and configured to alert medical personnel regarding the analyzed signals received from the environmental sensors or any changes performed by the microcontroller with regard to the first or second pumps.

29. (canceled)

30. The automated system of claim 27, wherein the enteral safe feeding catheter further comprises one or more optical fibers disposed within the first flexible wall and positioned along the axis, the optical fibers being configured to transmit light to a light emitter and imaging signals from an imaging sensor, wherein the light emitter and imaging sensor are disposed at the first distal end of the inner tube.

31. The automated system of claim 26, wherein the enteral safe feeding catheter further comprises a reporter disposed at the first distal end of the inner tube and configured to emit a detectable signal during insertion into a mammal, and the microprocessor is configured to detect the emitted signal.

32. A method of rapidly delivering liquid food or drugs to the stomach or duodenum of a mammal comprising:
(a) inserting the enteral safe feeding catheter of claim 1 into the esophagus of the mammal;
(b) advancing the first distal end of the inner tube into the stomach or duodenum until the second distal end of the outer tube engages a cardia of the stomach of the mammal; and
(c) delivering liquid food or drugs to the stomach or duodenum through the primary lumen.

33. The method of claim 32, further comprising monitoring one or more of pH, pressure, fluid flow, oxygen and moisture via environmental sensors disposed in the distal end of the one or more secondary lumens, wherein a change in pH activates a vacuum pump connected to the secondary lumens to aspirate reflux fluid from the esophagus.

34. The method of claim 33, wherein the change in pH simultaneously suspends flow of the liquid food or drugs within the inner tube.

35. (canceled)

36. The method of claim 33, wherein detection of an increase in pressure within the stomach or esophagus or detection of fluid flow within the esophagus activates the vacuum pump to aspirate reflux fluid from the esophagus and to suspend flow of food or drugs within the inner tube.

37. The method of claim 33, wherein the step of monitoring is accomplished by an electronic microcontroller in electrical communication with the one or more environmental sensors.

38. The method of claim 37, wherein the electronic microcontroller is configured to control the vacuum pump and to control delivery of the liquid food or drugs flowing within the inner tube.

39. The method of claim 32, wherein the step of inserting comprises detecting an emitted signal from a reporter disposed at the first distal end of the inner tube to monitor to the position of the catheter while advancing into the esophagus of the mammal.
